(19) United States
(12) Patent Application Publication
(10) Pub. No.: US 2012/0123489 A1
(43) Pub. Date: May 17, 2012

(54) ENERGY STORAGE ELEMENT DESIGN AND CONFIGURATION FOR IMPLANTABLE INTRAVASCULAR DEVICE

(76) Inventors: Terrance Ransbury, Chapel Hill, NC (US); Robert M. Smith, Raleigh, NC (US); Kenneth C. Armstrong, Cary, NC (US); John Miller, Shaker Heights, OH (US)

(21) Appl. No.: 13/165,585

(22) Filed: Jun. 21, 2011

Related U.S. Application Data
(60) Provisional application No. 61/356,896, filed on Jun. 21, 2010.
(60) Provisional application No. 61/368,890, filed on Jul. 29, 2010.
(60) Provisional application No. 61/441,495, filed on Feb. 10, 2011.

(51) Int. Cl. A61N 1/378; A61N 1/39; H05K 13/04; A61N 1/372

(52) U.S. Cl. 607/5; 607/72; 607/9; 607/45; 29/825

(57) ABSTRACT

An energy storage component for use with an implantable intravascular medical device that maximizes the useful volume available in the implantable intravascular medical device by providing a bore in a capacitor or battery that allows connections between various segments of the implantable intravascular medical device to be connected with one another.

### RELATED APPLICATIONS

The present application claims the benefit of U.S. Provisional Application Nos. 61/356,896, filed on Jun. 21, 2010; 61/368,890, filed on Jul. 29, 2010; and 61/441,495, filed on Mar. 11, 2011, the disclosures of each of which are hereby incorporated by reference in their entireties.

### FIELD OF THE INVENTION

The present invention generally relates to implantable medical devices. More particularly, the present invention relates to energy storage components such as batteries and capacitors, and their arrangement in implantable intravascular devices.

### BACKGROUND OF THE INVENTION

Implantable devices that provide long-term active therapies such as artificial pacemakers, defibrillators, and implantable cardioverter-defibrillators (“ICDs”) have been successfully implanted in patients for years for treatment of heart rhythm conditions. Pacemakers are implanted to detect periods of bradycardia and deliver low energy electrical stimuli to increase the heart rate. ICDs are implanted in patients to cardiovert or defibrillate the heart by delivering high-energy electrical stimuli to slow or reset the heart rate in the event a ventricular tachycardia (VT) or ventricular fibrillation (VF) is detected. Another type of implantable device detects an atrial fibrillation (AF) episode and delivers an electrical stimuli to the atria to restore electrical coordination between the upper and lower chambers of the heart. Neurostimulators deliver neuromodulation therapy to the nervous systems to treat a variety of symptoms and conditions, and can deliver electrical stimulation alone or in combination with drug therapy. The current configuration for all of these implantable devices are typically hockey puck-sized devices implanted under the skin that deliver electrical stimuli via one or more leads that are implanted at the stimulation site—cardiac rhythm management devices typically utilize a lead implanted in the heart, while neurostimulator devices use a lead implanted on or near one or more nerves.

Next-generation implantable intravascular devices (IIDs) take the form of elongated, flexible devices that are implanted within the vascular system of a patient, instead of subcutaneously as with conventional implantable devices. Examples of these implantable intravascular devices are described, for example, in U.S. Published Patent Application Nos. 2004/0249431, 2007/0255379, and 2008/0167702, and U.S. Pat. Nos. 7,082,336, 7,529,589, 7,617,007 and 7,925,352. These IIDs contain electric circuitry and/or electronic components that are hermetically sealed to prevent damage to the electronic components and the release of contaminants into the bloodstream.

Due to the length of these IIDs, which in some cases can be approximately 10-60 cm in length, the devices generally are designed to be flexible enough to move through the vasculature while being sufficiently rigid to protect the internal components. Sufficient flexibility has been accomplished by constructing the IID body from multiple elongate rigid or semi-rigid containers connected to one another with a flex coupling. Each container includes an interior space therein for housing functional components, such as batteries, capacitors, microprocessors, antennas, and associated circuitry for sensing, detection, therapy delivery, and telemetry. The components within respective containers must be electrically coupled to one another while maintaining flexibility of the device. In the case of an IID having three or more containers, electrical connections may need to be made between non-adjoining containers, increasing the complexity of the electrical connections due to routing connections through multiple containers of the IID.

One prior approach to the packaging and connecting of the various components within an IID is described in U.S. Patent Application No. 2007/0265673 and U.S. Pat. No. 7,529,589. As described therein, a flex circuit is disposed between the outer diameter of a component and the inner diameter of the container housing, while a complex arrangement of connectors, pins and caps are utilized to carry the electrical connections through the flex couplers and between the components of the device.

While this approach met the need of connecting the various components of the IID, an opportunity exists for a less complex and more space efficient solution.

### SUMMARY OF THE INVENTION

The present invention addresses the current need by providing energy storage components that maximize the use of the available space in the various containers comprising an implantable intravascular device. An energy storage component configured with a through-hole is contemplated, allowing the routing of electrical connectors between various components of the device, while maximizing the volume of the energy storage components resulting in a corresponding increase in charge capacity per unit length over previously known energy storage assemblies.

Embodiments of the present invention provide an alternative form factor for capacitors within an IID: that of a cylindrical container having an annular cross section and defining a through-hole forming a central passageway. The capacitor and its container are formed with a passage through the center of the entire length of the capacitor assembly, which provides space for the routing of conductors therein. By allowing passage of conductors through the center of the capacitor assembly, it is possible to expand the outer diameter of the capacitor to meet the inside diameter of the device enclosure. This relatively small expansion in capacitor diameter yields a significant increase in usable capacitor cross-section area, and thus improves capacitor volume per unit length. Greater volume per unit length can provide for a shorter capacitor, and thus a shorter IID without sacrificing electrical capacity. Length of the IID can be important to the physiological suitability of the device for a large patient population. Alternatively, without sacrificing electrical capacity or reducing the length of the IID, the IID could be provided with a smaller cross-sectional area. Generally, the shorter or smaller the device, the greater the number of patients that can have an appropriately size device implanted and receive therapeutic treatment.

In one embodiment, an implantable intravascular device is provided, having an elongate device body dimensioned for implantation in a vascular system of a patient. The elongate device body includes a proximal end, a distal end and a longitudinal axis extending therebetween. The implantable intravascular device further includes an elongated energy storage component including a bore aligned coaxially with at least a portion of the longitudinal axis of the device body, pulse generator circuitry electrically connected to the energy storage component, and at least one elongated electrical conductor connected to at least one of the energy storage component and the pulse generator circuitry and at least partially disposed within the central bore of the energy storage component.

In one embodiment, a method of manufacturing an implantable intravascular device is provided, the method comprising attaching a tensile member to a first compartment, and attaching an energy storage component within the first compartment, the energy storage component including a central bore, wherein the tensile member is routed through the central bore. The method further includes attaching a flex coupler to the first compartment, attaching a second compartment to the flex coupler, attaching pulse generator circuitry within the second compartment, electrically connecting the energy storage component to the pulse generator circuitry via at least one electrical conductor that is routed through the flex coupler and at least partially disposed within the central bore of the energy storage component, and attaching the tensile member to the second compartment.

### BRIEF DESCRIPTION OF THE DRAWINGS

The invention may be more completely understood in consideration of the following detailed description of various embodiments of the invention in connection with the accompanying drawings, in which:

FIG. 1 is a perspective view of an implanted intravascular device.

FIG. 2A is a perspective view of a prior art solid capacitor configuration with an external flex circuit.

FIG. 2B is an end view of the capacitor of FIG. 2A, disposed within an implantable intravascular device container.

FIG. 3A is a perspective view of a capacitor anode according to an embodiment of the invention.

FIG. 3B is a perspective view of a capacitor cathode according to an embodiment of the invention.

FIG. 3C is a perspective view of an annular cylindrical capacitor according to an embodiment of the invention.

FIG. 4A is a view of a capacitor anode according to an embodiment of the invention.

FIG. 4B is a cut-away view of a capacitor cathode according to an embodiment of the invention.

FIG. 4C is a cut-away view of a capacitor with a cathode-anode spacer according to an embodiment of the invention.

FIG. 4D is a cut-away view of a capacitor with a ceramic electrode-spacer according to an embodiment of the invention.

FIG. 4E is an external view of a capacitor according to an embodiment of the invention.

FIG. 5A is a side view of an energy storage component according to an embodiment of the invention.

FIG. 5B is a sectional view taken along line A-A of FIG. 5A.

FIG. 5C is an end view of FIG. 5A.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF THE EMBODIMENTS

In the following detailed description of the present invention, numerous specific details are set forth in order to provide a thorough understanding of the present invention. However, one skilled in the art will recognize that the present invention may be practiced without these specific details. In other instances, well-known methods, procedures, and components have not been described in detail so as to not unnecessarily obscure aspects of the present invention.

The present invention describes implantable intravascular devices and systems that may be used for a variety of functions to treat patients via electrical stimulation. These functions include defibrillation, cardioversion, and/or cardiac pacing. In general, the elements of an implantable intravascular device 100 (referred to herein as “IID” or “device”) for electrophysiological therapy include at least one elongate device body 104 and typically, but optionally, at least one lead 108 coupled to device body 104. Device body 104 includes a proximal end 102, a distal end 106, and optionally a tip portion 110. An anchoring arrangement 116 may be provided to secure IID 100 within the vasculature of a patient.

Additional information pertaining generally to the construction, arrangement and function of an IID suitable for use in accordance with the present invention can be found in U.S. Published Application Nos. 2004/0249431, 2007/0265673, 2008/0147168, 2008/0167702, 2009/0118798, 2009/0192579, 2009/0198251, 2010/0318166, and 2011/0004288, and U.S. Pat. Nos. 7,082,336, 7,363,082, 7,529,589, and 7,617,007, the disclosures of which are hereby incorporated by reference, other than claims and express definitions.

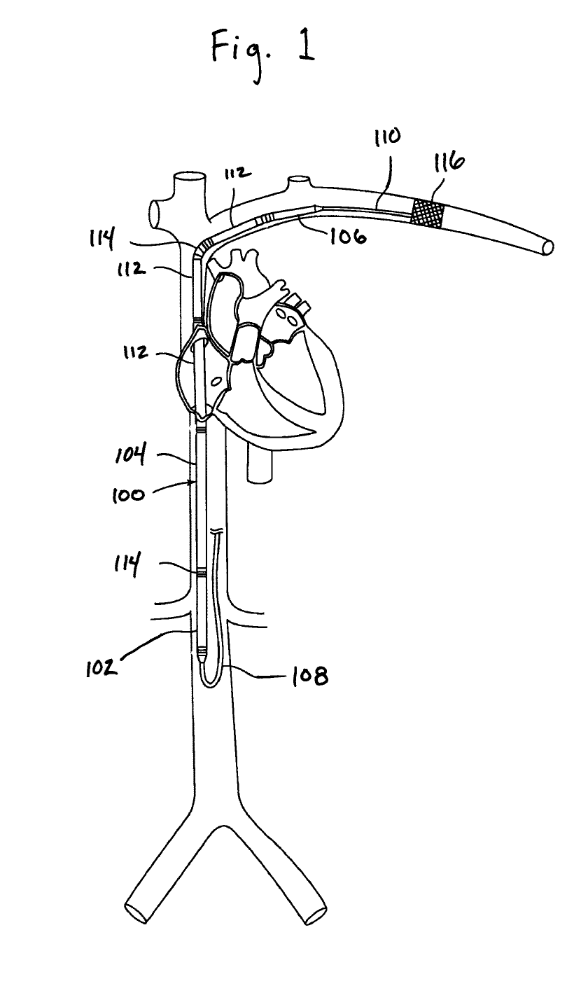

Referring to FIG. 1, IID 100 includes components, known in the art to be necessary to carry out the system functions of an implantable electrophysiology device. For example, IID 100 may include one or more pulse generators, including associated batteries, capacitors, microprocessors, communication circuitry and circuitry for generating electrophysiological pulses for defibrillation, cardioversion or pacing. The IID 100 may also include sense circuitry and detection circuitry for detecting arrhythmias or other abnormal activity of the heart. The specific components to be provided in the IID will depend upon the application for the device, and specifically whether the IID is intended to perform defibrillation, cardioversion, and/or pacing along with sensing functions.

In other embodiments, the IID can be used for diagnostic detection, nerve stimulation, and other patient treatments. Still another type of implantable device stores and delivers drug and/or gene therapies alone or in combination with electrical therapy, to treat a variety of conditions.

Implantable intravascular device 100 can be proportioned to be passed into the vasculature and to be anchored within the vasculature of the patient with minimal obstruction to blood flow. Suitable sites for introduction of the IID into the body can include, but are not limited to, the venous system using access through the right or left femoral vein or the right or left subclavian vein. In one embodiment, IID 100 can have a streamlined maximum cross sectional diameter which can be in the range of 3-15 mm or less, with a maximum cross-sectional diameter of 3-10 mm or less in one embodiment. The cross-sectional area of IID 100 in the transverse direction (i.e. transecting the longitudinal axis) can preferably be as small as possible while still accommodating the required components. This area can be in the range of approximately 79 mm̂2 or less, in the range of approximately 40 mm̂2 or less, or between 12.5-40 mm̂2, depending upon the embodiment and/or application.

Referring again to FIG. 1, device body 104 is comprised of one or more rigid or semi-rigid containers 112, or segments, joined together via flexible couplers 114. Containers 112 comprise a housing for the necessary components of IID 100. These containers 112 can be of any appropriate shape, cross-section, and length, but are depicted herein as having an elongate cylindrical shape with a diameter of approximately 3-15 mm and a length of approximately 20 mm to 75 mm. In order to allow for insertion of device body 104 having a plurality of rigid containers 112 into the vasculature, it can be desirable to limit the diameter to less than about 8 mm with a length of no more than about 70 cm. Elongate device body 104 is preferably dimensioned to have a length to diameter ratio of at least 10:1, up to 90:1.

Given the minimal space allowed for components, it can be desirable to arrange the device components within containers 112 so as to make efficient use of the available space. The length of containers 112 can vary, depending upon the ultimate implant location of each container 112 and the path through which each container must pass, as the amount of bending and varying size of the path can affect the maximum component size for different areas of the vasculature. Additional information pertaining to selecting and arranging appropriately-sized containers 112 can be found in U.S. Published Application No. 2010/0318166, incorporated by reference above.

The thickness of the walls of containers 112 also can vary, depending upon the application and the material being used. It can be desirable for the walls to be as lightweight as possible, while still providing for sufficient rigidity. In one example, container 112 can be made of a biocompatible material that is capable of sterilization and is conductive, with a sidewall thickness on the order of about 0.001″ to 0.005″. Possible materials include titanium, nitinol, stainless steel, nickel, or alloys thereof, as well as polymers such as nylon or polyurethane. The sidewall thickness can vary among containers 112, as well as within an individual container 112 in order to accommodate the internal components, etc.

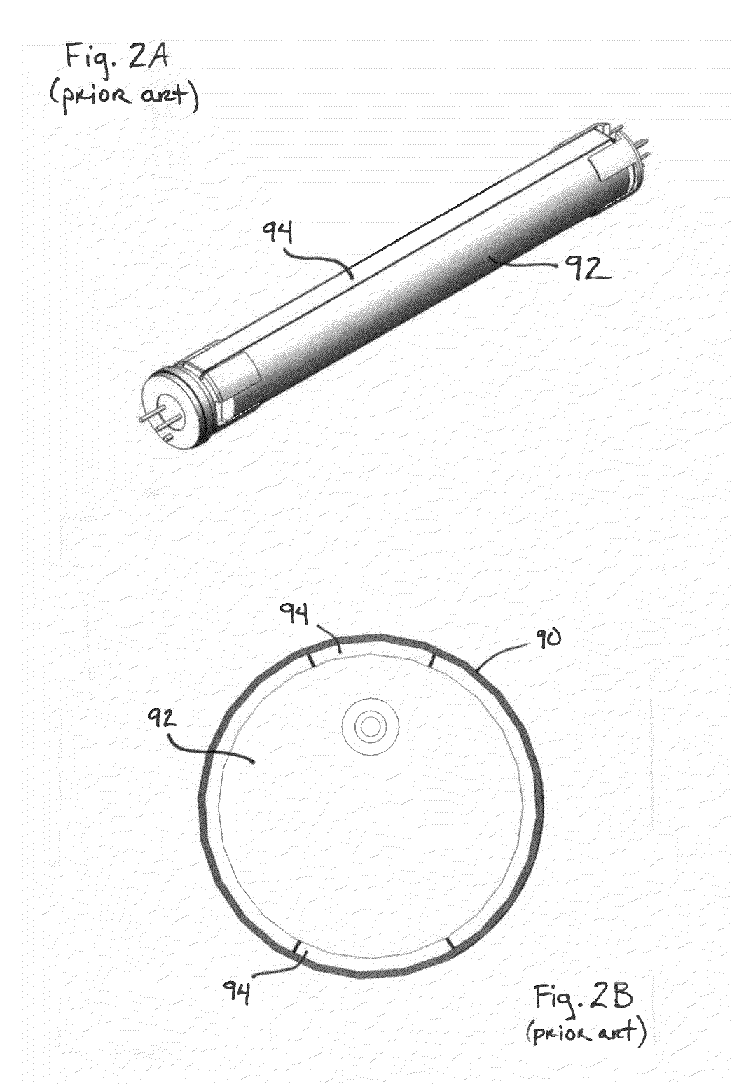

One challenge of IID construction is the sequencing and connection of the various components within the device body. For components such as circuitry, the routing of necessary connections has been relatively straightforward. For energy storage components such as batteries and capacitors, prior configurations of implantable intravascular devices utilized flex circuitry 94 disposed between the inner diameter of a container 90, and the outer diameter of the energy storage component 92 within the container 90. The energy storage components 92 were designed to be slightly smaller than the inside diameter of the container 90, leaving a small space to route the necessary conductors, typically in the form of flex circuits 94. Such an arrangement is depicted in FIGS. 2A-2B, as well as in U.S. Published Patent Application No. 2007/0265673. Additionally, complex intricate connections were required between adjacent containers, examples of which are also depicted and described in U.S. Published Patent Application No. 2007/0265673.

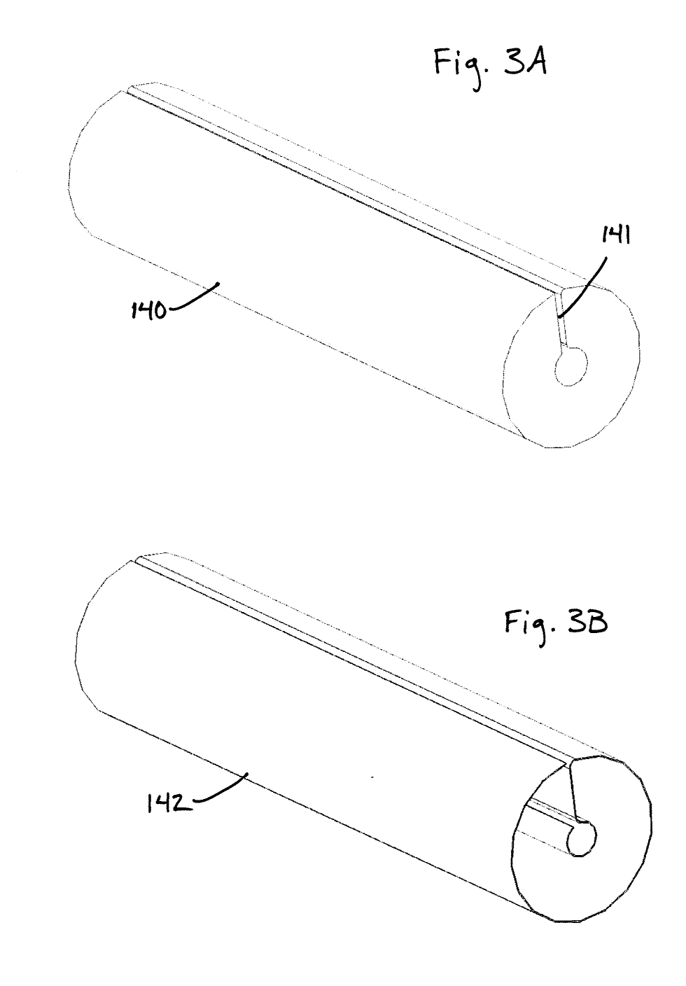

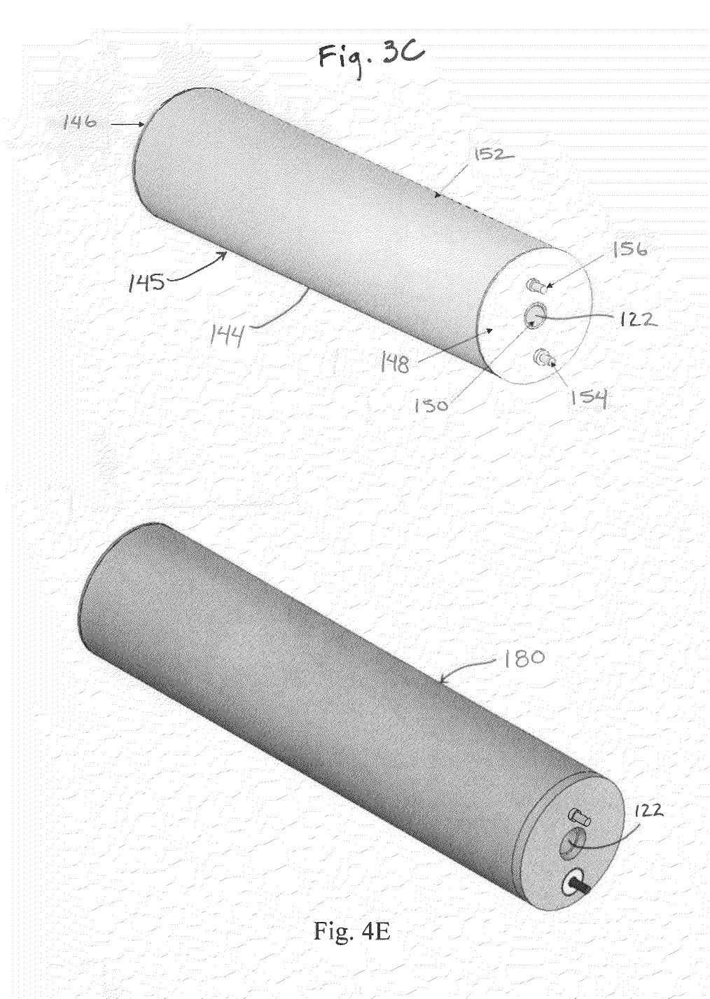

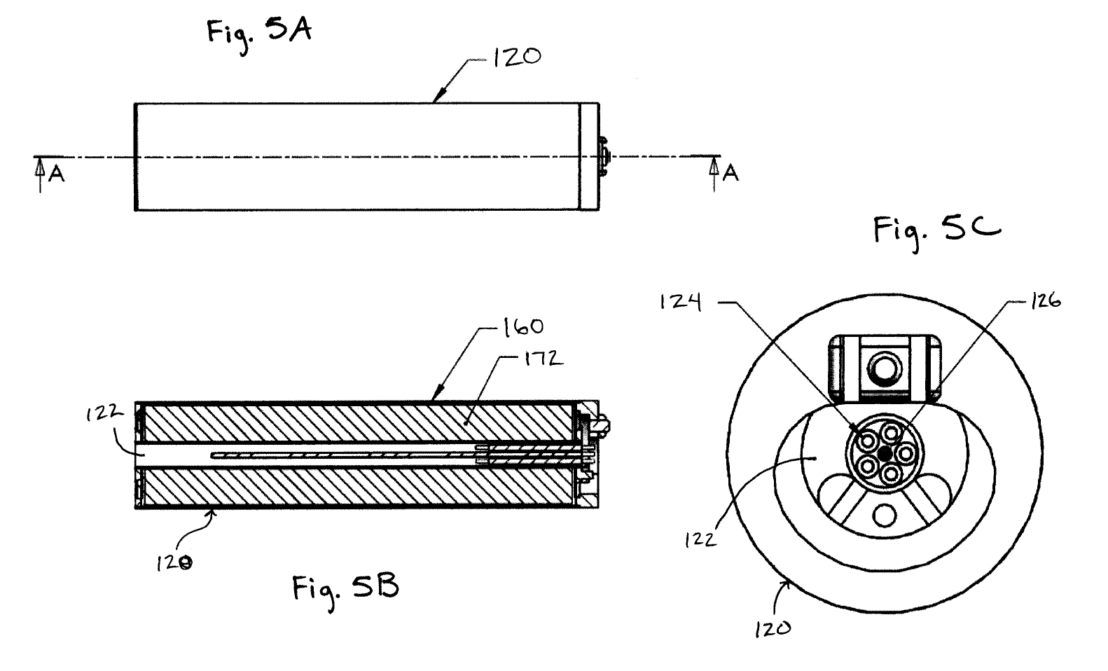

Referring now to FIGS. 3A-5C, an improved energy storage component 120 is depicted, which may comprise a battery or a capacitor. Energy storage component 120 includes a bore 122 which extends the entire length of component 120. In an embodiment where energy storage component 120 is cylindrical, bore 122 may be coaxial with a longitudinal axis of component 120. Bore 122 may also be located anywhere within energy storage component 120 for both cylindrical and non-cylindrical configurations of energy storage component 120. Bore 122 provides a path for the routing of connection means 124 through energy storage component 120, as well as a path for an internal tensile member 126. Bore 122 may also provide a path for routing of necessary conductors for lead 108.

An energy storage component 120 according to the present invention represents an improvement in usable space within a container 112 of IID 100. As discussed above, prior IID configurations routed the necessary electrical connections between the outer diameter of an energy storage component and the inner diameter of a container in which it was placed. Providing space for these electrical connections in such an arrangement limited the maximum allowable diameter of energy storage components. In an example prior IID configuration having a container inner diameter of 0.30 inches, an energy storage component may have had a diameter of 0.28 inches in order to leave space for necessary electrical connections. Such an arrangement resulted in an energy storage component with a cross-sectional area of approximately 0.062 square inches.

In contrast, an energy storage component 120 having a bore 122 through which electrical connections can be passed, can be sized to take advantage of the maximum allowable space within a container 112. For example, with a container 112 having an inner diameter of 0.30 inches, energy storage component 120 may be sized to have an outer diameter of approximately 0.30 inches, and an example bore diameter of 0.060 inches. Such configuration yields a cross-sectional area of approximately 0.068 inches, an almost ten percent improvement over the prior configuration. Energy storage component 120 can therefore be reduced in length as compared to prior configurations while providing the same amount of energy storage, thereby reducing the overall length of IID 100. Energy storage component 120 may be dimensioned to have a length to diameter ratio of at least 3:1, up to 25:1.

One example embodiment of energy storage component 120 comprises a wet tantalum slug capacitor 145. In one embodiment, capacitor assembly begins with the anode 140, which is formed as a cylinder with a longitudinal slit 141. The anode 140 is depicted in FIG. 3A. The anode 140 is encased in a porous separator (not depicted) to prevent contact with the cathode 142, and the cathode 142, depicted in FIG. 3B, is installed around the anode and separator. Cathode 142 is configured to provide an exposed surface adjacent to all of the core anode 140 faces. Cathode 142 is also formed to act as a spring, so that it will expand radially to contact the inside surface of the capacitor sleeve and provide an electrical connection. The anode 140, separator, and cathode 142 slide into capacitor enclosure 144, which is then filled with electrolyte and sealed. A complete capacitor component 145 is depicted in FIG. 3C.

The capacitor enclosure 145 is assembled from two end caps (bottom 146 and top 148), a center sleeve 150, and an outer sleeve 152. The anode connection pin 154 and cathode connection pin 156 protrude from the top cap 148. The outer sleeve 152, or case, of the capacitor is electrically connected to the cathode 142, and so the cathode pin 156 is welded to the enclosure 144. The anode pin 154 is electrically connected to the anodized tantalum slug anode 140 inside of the enclosure 144, so as to be insulated from the top cap 148 as it passes through. Capacitor hermeticity is maintained via seam welds between the enclosure components, and a glass-to-metal seal between the anode pin 154 and the top cap 148.

The glass-to-metal seal between the anode pin 154 and top cap 148 is formed prior to installation of the separator and cathode around the anode. The bottom cap 146 and sleeves can be welded together separately, prior to insertion of the anode/cathode assembly. The top cap welds occur as a final step, since the top cap is attached to the anode/cathode assembly.

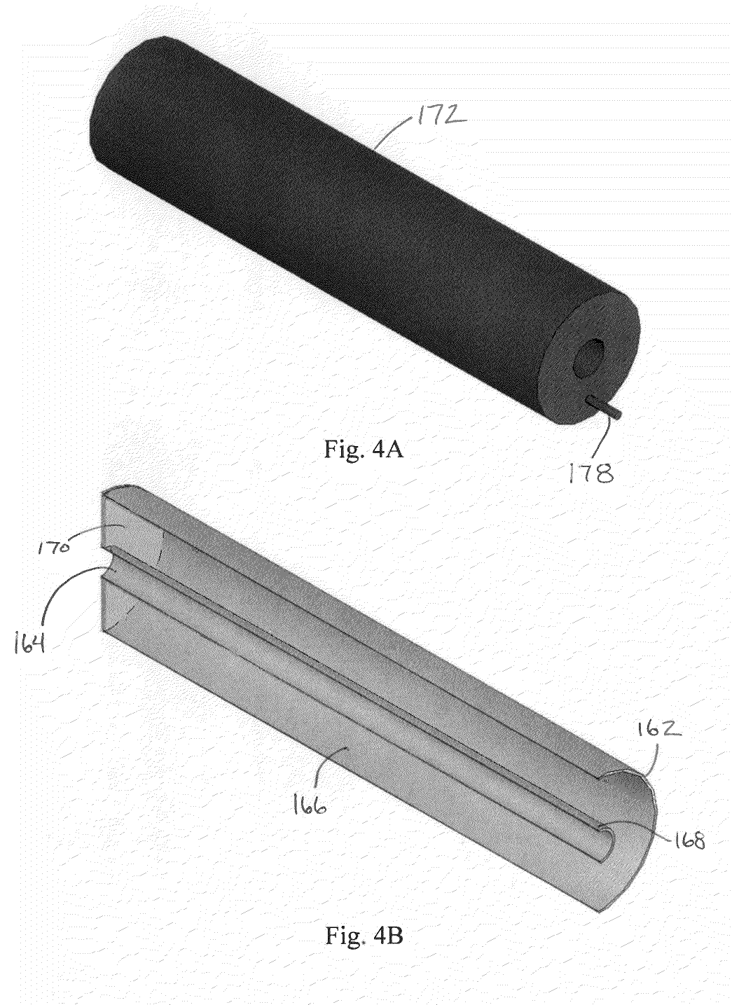

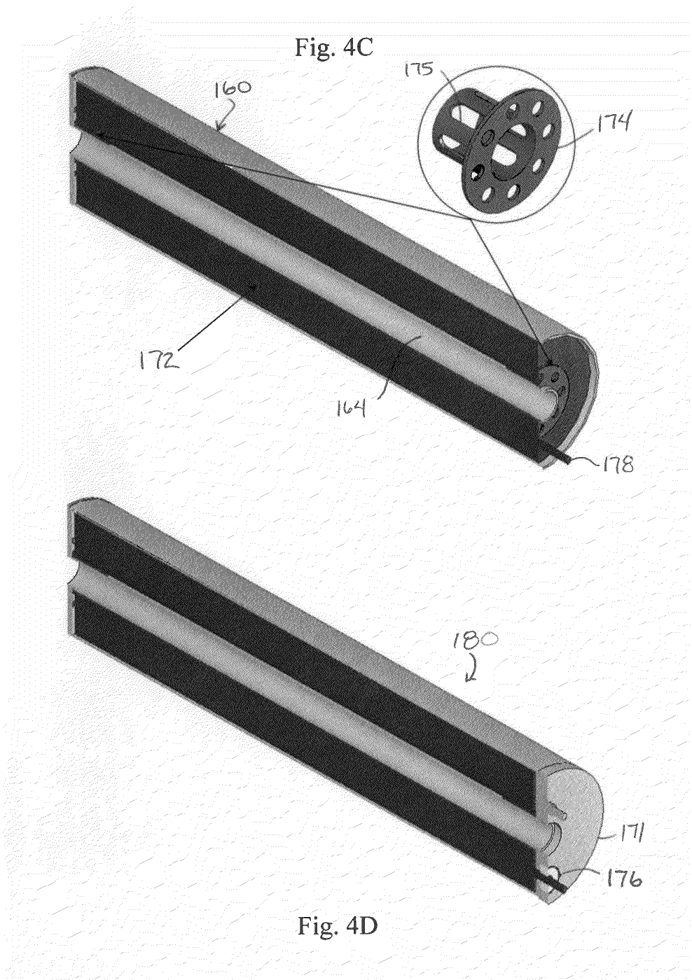

Referring now to 4A-4E, another embodiment of an energy storage component 120 is depicted. Capacitor 180 comprises an outer enclosure 160, whose inner surfaces act as the capacitor cathode. The enclosure 160 can be comprised of two nested titanium tubes 162, 164, and two titanium end caps 170, 171. The inside surface 166 of the outer enclosure tube 162 is coated with a pseudo-capacitive material, as is the outer surface 168 of the inner enclosure tube 164. The enclosure end caps 170, 171 are welded, or otherwise sealed, to the ends of the enclosure tubes 162, 164. A cross-section of the two enclosure tubes 162, 164 and a bottom cap 170 is depicted in FIG. 4B.

The anode 172 can be formed from sintered, anodized tantalum and takes the shape of an elongated annulus, or hollow cylinder. An exemplary anode 172, depicted in FIG. 4A, does not necessarily require the slit 141 of the anode 140 depicted in FIG. 3A. The anode 172 of FIG. 4A can be fitted with separators 174 to prevent it from contacting the enclosure and is inserted into the partially assembled enclosure 160 of FIG. 4B. The separators 174 can incorporate cutouts 175 to allow ion flow between adjacent portions of anode 172 and enclosure 160. An example cross-sectional view of the assembled anode 172, separators 174, and enclosure 160 are shown in FIG. 4C, which further includes a magnified view of one of the separators 174 with cutouts 175.

The enclosure 160 is filled with electrolyte, and the top cap 171 is installed and welded to the inner tube 164 and outer tube 162. A ceramic spacer 176 is brazed into the top cap 171 to admit the anode pin 178 through the cap 171, while providing electrical isolation from enclosure 160 (i.e., the cathode). The pin 178 can be brazed to the inside diameter of the ceramic spacer 176 to seal the capacitor 180 and contain the electrolyte. A cross section of the completed capacitor 180 is depicted in FIG. 4D, and a perspective view is depicted in FIG. 4E.

The capacitor 180 can be of a hybrid wet tantalum electrolytic type. A sintered tantalum anode is oxidized to form a Ta₂O₅ dielectric layer, and is flooded with electrolyte. The cathode is formed from a thin tantalum sheet coated with a ruthenium-oxide film. This capacitor is electrochemically identical to existing capacitors used in IIDs, but differs in the physical details necessary to realize its unique form factor. Other known capacitor anode, cathode, and dielectric materials can be substituted to improve or modify the electrical performance of the capacitor. For example, the polymer polyvinylideve floride (PVDR) can be combined with polymer CTFE to form a dielectric material. Other examples of electrolytic capacitor components can be found in U.S. Pat. Nos. 4,434,084, 4,523,255, 5,098,485, and 7,511,943, which are incorporated herein in their entirety by reference. Examples include the use of titanium-oxide, silver, carbon or lithium-ion nano-tubes in capacitor components as understood by those skilled in the art.

In order to provide bore 122 within a capacitive energy storage component 120, the energy storage component may be formed around a plug during manufacturing. For example, in the case of a rolled (or wound) construction capacitor, having a rolled foil (or film) anode, the capacitor may be constructed by rolling the film around a cylindrical form, resulting in a bore 122 within capacitive energy storage component 120. In the case of a formed capacitor, such as solid tantalum capacitors, the slug is formed around a cylindrical tube serving as an anode or cathode depending upon the design.

In one embodiment, one or more energy storage components 120 according to the present invention may be used to construct an implantable intravascular defibrillator. Such a device would typically require at least one capacitor, and at least one battery, in addition to associated circuitry for providing defibrillation therapy.

In another embodiment, one or more energy storage components 120 according to the present invention may be used to construct an implantable intravascular pacing device. Such a device would typically require at least one battery, in addition to associated circuitry for providing artificial pacing.

In another embodiment, a method of assembling an IID 100 having one or more energy storage components 120 is provided. Generally, implantable intravascular device 100 comprises a device body 104 having a plurality of containers 112 joined by flexible couplers 114, and optionally one or more leads 108 coupled to or integrated with device body 104. A first container 112 is provided, and enclosed on one end such as by welding of a cap onto the end. A tensile member 126 is secured inside container 112 by suitable means such as welding, clamping, or mechanical fastening. Tensile member 126 is constructed of a material having a high tensile strength, yet is sufficiently flexible to be suitable for use in an IID. In one embodiment, tensile member 126 is constructed of MP35N alloy. Tensile member 126 is intended to extend the entire length, or substantially the entire length, of device body 104, and provides additional strength to IID 100 which can be useful in the event IID 100 needs to be explanted from a patient.

One or more components are then inserted into the space within container 112. The component(s) may be one or more of a battery, a capacitor, microprocessor, antenna, or associated circuitry for sensing, arrhythmia detection, therapy delivery, and telemetry. Components such as circuitry, microprocessors or antennas may be arranged around tensile member 126 within container 112, or may be configured to include a bore or passageway through which tensile member 126 can be routed. Components such as batteries or capacitors, as described herein, are provided with a bore 122 such that the component is advanced over tensile member 126. The one or more components are then secured in place as needed within container 112. One or more connection means 124 are attached to the one or more components, and routed in any suitable space for components such as circuitry, microprocessors or antennas, or routed in bore 122 for energy storage components such as batteries or capacitors. An end cap may be provided for container 112 if needed, with appropriate pass-through sealing for connection means 124. Components may also be pre-assembled into containers prior to final assembly of IID 100.

A flex coupler 114a is then attached to container 112, and a second container 112 may be advanced over tensile member 126 and secured to flex coupler 114. Additional information pertaining to the configuration, construction and assembly of flexible couplers suitable for use with the present invention may be found in U.S. Pat. No. 7,363,082.

The assembly of IID 100 continues with additional components housed in additional containers which are joined by additional flex couplers, until the desired device configuration is obtained. Connection means 124 are routed through flex couplers 114 and bores 122 and to the various components as needed. One advantage of the present invention over prior approaches is that complex wire interconnects are not needed. Rather, connection means 124 are free to pass directly through flex couplers 114. When assembling the final container 112 of a device, tensile member 126 is cut to a desired length and secured within the final container. Additional features, such as one or more leads 108, or tip portions or anchor portions may also be assembled and connected as needed. The above series of steps is illustrative, and do not necessarily need to be performed in the order they are listed.

In one embodiment, instructions for implanting IID 100 in accordance with the various embodiments described herein in the form of printed or electronically, optically or magnetically stored information to be displayed, for example, are provided as part of a kit or assemblage of items prior to surgical implantation of device 100. Instructions for implanting device 100 in accordance with the various embodiments described herein may be provided, for example, by a manufacturer or supplier of IID 100 separately from providing the device, such as by way of information that is accessible using the Internet or by way of seminars, lectures, training sessions or the like. Further, instructions for manufacturing IID 100 may also be provided.

For clarity, certain terms used throughout this specification are defined as follows, unless expressly provided otherwise. “Component” includes the necessary elements of an operational IID, typically residing within a container, and may be referring to a battery, capacitor, microprocessor, antenna, or associated circuitry for sensing, arrhythmia detection, therapy delivery, and telemetry. “Container”, when used in reference to a segment of the device body, is intended to refer to a portion of the device body configured to securely and safely retain a component therein, and may alternatively be referred to as an enclosure, compartment, segment, housing segment, housing portion, or rigid container. “Electrical connection means” is intended to refer to one or more of conductors, wires, cables, flex circuits, conduits, tubing, or other such connectors necessary for and associated with the operational coupling of the various components contained within an IID. “Energy storage component” is intended to refer to a battery or capacitor, and may alternatively be referred to as an energy storage element or energy storage unit. “Pulse generator” is intended to refer to the IID device body, including circuitry, microprocessors, one or more energy storage components (e.g., batteries, capacitors), housing containers, flex couplers, and electrical connections. “Pulse generator circuitry” is intended to refer to the circuitry associated with one or more of sensing, detecting, and delivering therapy pulses.

Many of the device configurations, components, retention devices and methods, implantation methods, and other features depicted and described herein can be used with other forms of intravascular implants. Such implants might include, for example, artificial pancreas implants, diagnostic implants with sensors that gather data such as properties of the patient's blood (e.g., blood glucose level), and/or devices that deliver drugs or other therapies into the blood from within a blood vessel.

Various embodiments of systems, devices and methods have been described herein. These embodiments are given only by way of example and are not intended to limit the scope of the present invention. It should be appreciated, moreover, that the various features of the embodiments that have been described may be combined in various ways to produce numerous additional embodiments. Moreover, while various materials, dimensions, shapes, implantation locations, etc. have been described for use with disclosed embodiments, others besides those disclosed may be utilized without exceeding the scope of the invention.

Persons of ordinary skill in the relevant arts will recognize that the invention may comprise fewer features than illustrated in any individual embodiment described above. The embodiments described herein are not meant to be an exhaustive presentation of the ways in which the various features of the invention may be combined. Accordingly, the embodiments are not mutually exclusive combinations of features; rather, the invention may comprise a combination of different individual features selected from different individual embodiments, as understood by persons of ordinary skill in the art.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims for the present invention, it is expressly intended that the provisions of Section 112, sixth paragraph of 35 U.S.C. are not to be invoked unless the specific terms “means for” or “step for” are recited in a claim.

What is claimed is:

1. An implantable intravascular device, comprising:
an elongate device body dimensioned for implantation in a vascular system of a patient, the elongate device body having: a proximal end, a distal end and a longitudinal axis extending therebetween;
an elongated energy storage component including a bore aligned coaxially with at least a portion of the longitudinal axis of the device body;
pulse generator circuitry electrically connected to the energy storage component; and
at least one elongated electrical conductor connected to at least one of the energy storage component and the pulse generator circuitry and at least partially disposed within the bore of the energy storage component.

2. The implantable intravascular device of claim 1, wherein the longitudinal axis is non-linear and the elongated device body includes a plurality of compartments flexibly coupled end to end along the longitudinal axis and the energy storage component and the pulse generator circuitry are each disposed within a compartment.

3. The implantable intravascular device of claim 2, wherein the at least one elongated electrical conductor is routed through a flex coupler that flexibly couples adjacent compartments along the longitudinal axis.

4. The implantable intravascular device of claim 1, further comprising a lead attached to one of the proximal end or the distal end.

5. The implantable intravascular device of claim 1, wherein the implantable intravascular device comprises an artificial cardiac pacemaker and the energy storage component comprises a battery.

6. The implantable intravascular device of claim 1, wherein the implantable intravascular device comprises an implantable cardioverter-defibrillator and the energy storage component comprises a capacitor.

7. The implantable intravascular device of claim 1, wherein the implantable intravascular device comprises a neurostimulator and the energy storage component comprises a battery.

8. The implantable intravascular device of claim 1, further comprising an elongated tensile member disposed within the device body and routed through the bore of the energy storage component, the tensile member extending between the proximal end and the distal end.

9. The implantable intravascular device of claim 1, wherein the energy storage component has a length to diameter ratio of at least 3:1.

10. The implantable intravascular device of claim 1, wherein the elongate device body has a length to diameter ration between 10:1 and 90:1.

11. A method of manufacturing an implantable intravascular device, comprising:
attaching a tensile member to a first compartment;
attaching an energy storage component within the first compartment, the energy storage component including a central bore, wherein the tensile member is routed through the central bore;
attaching a flex coupler to the first compartment;
attaching a second compartment to the flex coupler;
attaching pulse generator circuitry within the second compartment;
electrically connecting the energy storage component to the pulse generator circuitry via at least one electrical conductor that is routed through the flex coupler and at least partially disposed within the central bore of the energy storage component; and
attaching the tensile member to the second compartment.
